(19) United States
(12) Patent Application Publication
(10) Pub. No.: US 2019/0216645 A1
(43) Pub. Date: Jul. 18, 2019

(54) SURGICAL DEVICE FOR AB-EXTERNO SUB-RETINAL FLUID DRAINAGE

(71) Applicant: THE REGENTS OF THE UNIVERSITY OF CALIFORNIA, Oakland, CA (US)

(72) Inventors: Michael Adrian Klufas, Far Hills, NJ (US); Jean-Pierre Hubschman, Beverly Hills, CA (US); Rory Geoghegan, Los Angeles, CA (US)

(21) Appl. No.: 16/335,813

(22) PCT Filed: Sep. 22, 2017
(86) PCT No.: PCT/US17/52929

§ 371 Date: Mar. 22, 2019

Related U.S. Application Data
(60) Provisional application No. 62/398,778, filed on Sep. 23, 2016.

(51) Int. Cl. A61F 9/007; A61B 90/30

(52) U.S. Cl. CPC A61F 9/00727; A61B 2017/00867; A61B 90/30; A61F 9/00736

(57) ABSTRACT

The present invention provides improved surgical devices for ab-externo sub-retinal fluid drainage and methods for using the same. The surgical devices are illuminated, allow for a reinforced mark to be placed on the sclera, feature a deployable needle, and enable direct visualization of retinal pathology via transillumination of the sclera. The surgical devices allow more precise localization of the site of retinal tears and subretinal fluid drainage.

### CROSS-REFERENCE TO RELATED APPLICATIONS

This application claims priority to U.S. Provisional Patent Application No. 62/398,778, filed Sep. 23, 2016, the contents of which are incorporated by reference herein in its entirety.

### BACKGROUND OF THE INVENTION

Despite an increased trend toward pars plana vitrectomy (PPV) as a primary treatment for retinal detachment (RD) repair, scleral buckling remains a mainstay treatment for young, phakic (no history of cataract surgery) patients with retinal detachment as well as other conditions such as retinal dialysis (specific type of break in the retina particularly amenable to scleral buckling). Significant factors contributing to the decline of buckling include few advances in surgical techniques and the lack of any improvement in the instrumentation for scleral buckling over the past four decades.

External drainage methods of subretinal fluid during RD repair are also in need of improvement. Current methods include cutting through scleral fibers with a sharp blade and entering the subretinal space with a needle, or directly piercing the sclera with a needle and entering the subretinal space. The shortcomings of these techniques include: the location of the drainage site cannot be directly visualized; these techniques do not allow for controlled drainage of subretinal fluid by the surgeon; and they do not decrease the risk of hemorrhage at the site of drainage when the subretinal space is entered.

There is a need in the art for an improved device for use in RD repair. The present invention meets this need.

### SUMMARY OF THE INVENTION

In one aspect, the present invention relates to a surgical device comprising: an elongate shaft having a longitudinal axis, a posterior end, and an anterior end having an aperture that is recessed and encircled by a perimeter, wherein the anterior end and the aperture are curved away from the longitudinal axis at an angle of at least 30°; a light source positioned within the aperture; an actuator positioned along the length of the shaft; and a needle connected to the actuator; wherein the needle tip is extendable out of the aperture via activation of the actuator.

In one embodiment, the angle is between 30° and 120°. In one embodiment, the angle is 90°. In one embodiment, the perimeter forms a blunt scleral depressor. In one embodiment, the device further comprises a reservoir in the shaft connected to a delivery port near the aperture, wherein the reservoir stores a material selected from the group consisting of: an ink, a resin, and a polymer, and wherein the delivery port ejects the material stored in the reservoir.

In one embodiment, the device further comprises connection means to a source of power. In one embodiment, the source of power is a battery in the shaft. In one embodiment, the source of power is external to the device. In one embodiment, the device further comprises diathermy means near the aperture. In one embodiment, the device further comprises cautery means near the aperture.

In one embodiment, the light source is an optical fiber having attachment means to an external source of light. In one embodiment, the light source is at least one light emitting diode (LED) or laser diode. In one embodiment, the light source is connected to a controller. In one embodiment, the controller controls light activation, intensity, focus, diffusion, frequency, strobing, and color.

In one embodiment, the needle is constructed from a malleable material. In one embodiment, the malleable material is nitinol. In one embodiment, the connection between the needle and the actuator is adjustable by a locking member. In one embodiment, the needle is removable.

In one embodiment, the device further comprises a lumen having an anterior end near the aperture and a posterior end connected to a fluid pump. In one embodiment, the fluid pump is controllable to generate a positive pressure, a negative pressure, or both. In one embodiment, the lumen is positioned within the needle.

### BRIEF DESCRIPTION OF THE DRAWINGS

The following detailed description of exemplary embodiments of the invention will be better understood when read in conjunction with the appended drawings. It should be understood, however, that the invention is not limited to the precise arrangements and instrumentalities of the embodiments shown in the drawings.

FIG. 1 depicts a perspective view of an exemplary surgical device.

FIG. 2A through FIG. 2C depict a cross section view of an exemplary surgical device in a retracted configuration.

FIG. 3A through FIG. 3C depict a cross section view of an exemplary surgical device in a deployed configuration.

FIG. 4A depicts an exemplary prototype surgery device next to a typical depressor with a light source illuminating the optical fiber of the prototype device.

FIG. 4B depicts the needle puncture size of an exemplary prototype surgery device.

FIG. 5 depicts an experimental setup using an exemplary prototype surgery device to illuminate a porcine eye with a fundus lens to look into the eye. The prototype includes an aperture angled at about 90 degrees, enabling the operator to reach the rear of the eye while holding the device in a relaxed grasp.

FIG. 6 depicts the results of experiments performed on a porcine eye using an exemplary prototype surgery device. The top bracket points to a circular indentation with ink markings left by the blunt perimeter of the device. The bottom bracket points to a series of test needle punctures.

FIG. 7 depicts the results of additional experiments performed on a porcine eye using an exemplary prototype surgery device. Visible on the scleral surface are two indentations and a needle puncture in the center of each.

FIG. 8 depicts the view through a fundus lens of an experiment illuminating and puncturing a porcine eye using an exemplary prototype surgery device. The circled area indicates the illumination provided by a light source illuminating the optical fiber of the prototype device. The arrow points to the needle within the eye reflecting light from the optical fiber. The additional two light points are reflections of operating room lights on the fundus lens.

### DETAILED DESCRIPTION

The present invention provides improved surgical devices for ab-externo sub-retinal fluid drainage and methods for using the same. The surgical devices are illuminated, allow for a reinforced mark to be placed on the sclera, feature a deployable needle, and enable direct visualization of retinal pathology via transillumination of the sclera. The surgical devices allow more precise localization of the site of retinal tears and subretinal fluid drainage.

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

### Surgical Devices

The present invention provides surgical devices for identifying retinal breaks and for draining subretinal fluid, which are critical steps of retinal detachment repair. The surgical devices are able to mark the sclera to indicate the external location of a retinal break. The surgical devices are also able to enter the subretinal space under direct visualization using a retractable needle.

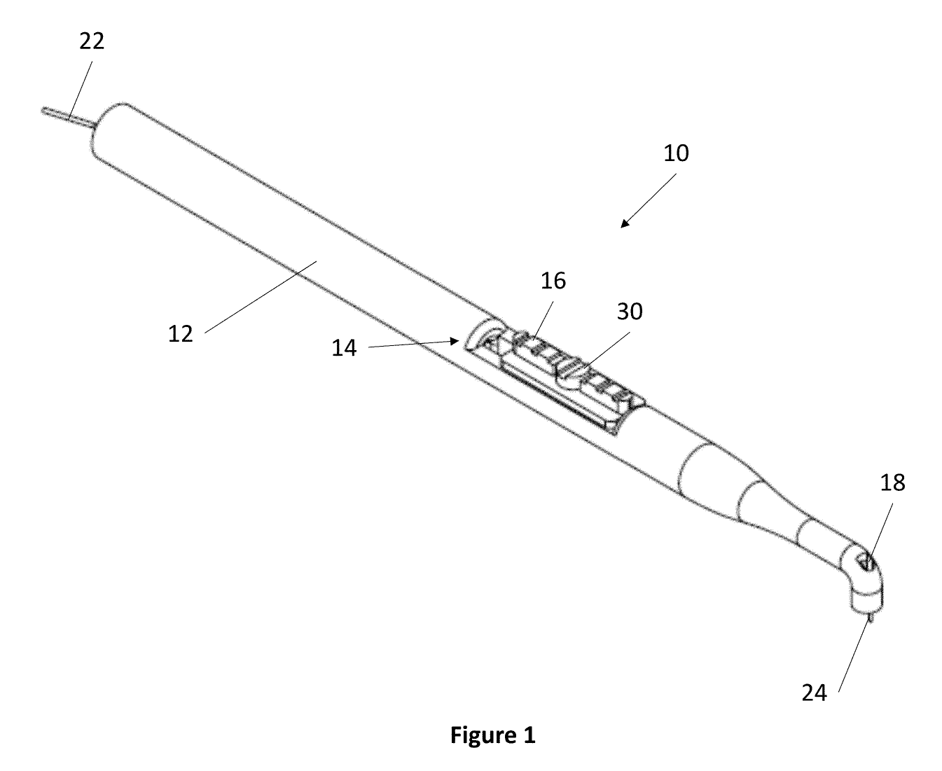

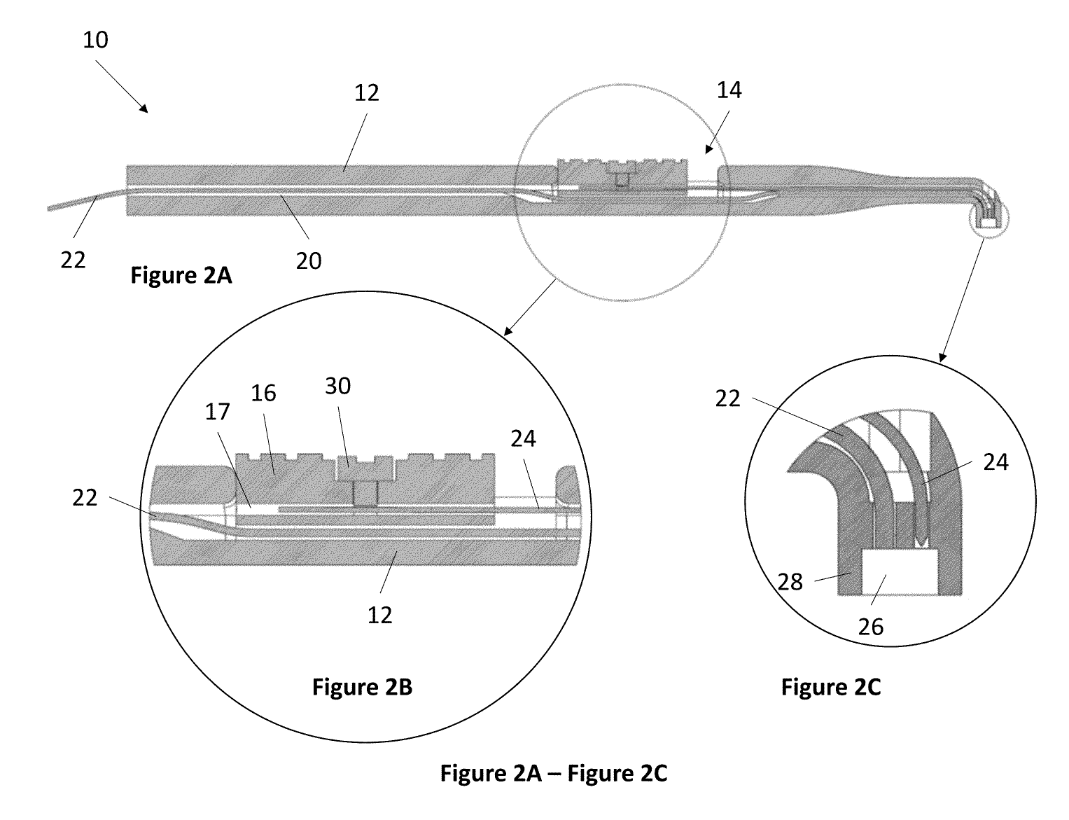

Referring now to FIG. 1, an exemplary device 10 is depicted. Device 10 comprises an elongate shaft 12 having a longitudinal axis, an anterior end, and a posterior end. Referring now to FIG. 2A through FIG. 2B, the interior components of device 10 are visible. Shaft 12 comprises a cutout 14 through which actuator 16 is accessible. Actuator 16 comprises actuator lumen 17 and locking member 30. The anterior end of shaft 12 comprises aperture 26. In certain embodiments, the anterior end of shaft 12 is curved, such that aperture 26 is curved away from the longitudinal axis of shaft 12 at an angle. The angle can be any angle between 30° and 120°. In one embodiment, the angle is 90°. A curved embodiment permits a user to point the anterior end of shaft 12 at difficult to reach areas, such as certain areas of the eye (including the anterior and posterior sclera) and orbit during an ophthalmic surgery procedure.

Aperture 26 is recessed into the anterior end of shaft 12, such that aperture 26 is encircled by a continuous perimeter 28. In some embodiments, perimeter 28 is blunt, such that it can be used as a scleral depressor for marking purposes by manual force with secondary temporary desiccation of the sclera without injuring the eye or inadvertently perforating the sclera. In other embodiments, aperture 26 further comprises one or more means for applying a marking (not shown), such as a delivery port positioned near aperture 26 for ejecting a marking substance such as a colored ink, resin, polymer, and the like. The marking substance can be stored in a reservoir fluidly connected to the delivery port, the reservoir being interior to or exterior to shaft 12. In other embodiments, aperture 26 further comprises one or more means for applying heat, such as by cautery or diathermy. The means for applying heat can be a metal tip or filament for cautery, or one or more electrodes for monopolar or bipolar electrocautery. In some embodiments, the means for applying heat are channeled through the needle 24. In some embodiments, the means for applying heat are embedded on the blunt face of perimeter 28. In such embodiments, the means for applying heat are connected to a power source, such as a battery within shaft 12 or an external power source.

A lumen 20 extends from the posterior end of shaft 12 through the anterior end of shaft 12, wherein lumen 20 opens into aperture 26. Optical fiber 22 is positioned within lumen 20, wherein optical fiber 22 extends out of the posterior end of shaft 12 and terminates at aperture 26. In various embodiments, optical fiber 22 comprises an attachment means, such as any suitable optical fiber connector known in the art, to connect to an external source of light to provide a means of illumination. For example, vitreoretinal surgery machines typically used in the art to provide light to vitrectomy light pipes may be connected to optical fiber 22 to provide illumination. In certain embodiments, the means of illumination may be separately provided or supplemented with at least one light emitting diode (LED) or laser diode positioned near aperture 26. The at least one LED or laser diode can further be coupled to a controller to adjust light color, intensity, focus/diffusion, frequency, strobing, and the like. In various embodiments, device 10 further comprises a switch for controlling the activation, light color, intensity, focus/diffusion, frequency, and strobing of the external or internal means of illumination (not shown).

Needle 24, having a posterior end and an anterior end, is positioned within lumen 20. In various embodiments, needle 24 is positioned such that the posterior end is within actuator lumen 17 and the anterior end is near aperture 26. The posterior end of needle 24 is fixable within actuator lumen 17 by locking member 30. For example, locking member 30 may be engaged to fasten the posterior end of needle 24 within actuator lumen 17, such that sliding actuator 16 accordingly slides needle 24. Alternatively, locking member 30 may be disengaged to unfasten the posterior end of needle 24 from actuator lumen 17, such that sliding actuator 16 does not cause any movement in needle 24. Accordingly, locking member 30 may be engaged and disengaged to adjust the position of needle 24, or disengaged to remove needle 24. Needle 24 may be inserted into and removed from device 10 by way of opening 18. In certain embodiments, device 10 may be constructed with needle 24 embedded within actuator 16, wherein needle 24 is secured by a compound such as glue or epoxy, or wherein needle 24 is secured by press-fitting or molding actuator 16 around the posterior end of needle 24. In embodiments where device 10 is constructed with needle 24 embedded, device 10 may also be constructed without locking member 30 or opening 18.

In some embodiments, needle 24 is constructed from a malleable material, such that needle 24 may navigate through the lumen of a curved shaft 12. In certain embodiments, the malleable material is nitinol. In other embodiments, the malleable material can comprise flexible polymers (such as polyethylene and polypropylene) and metals (such as aluminum and stainless steel).

In some embodiments, device 10 includes fluid transfer means. For example, in some embodiments device 10 includes a cannula having an anterior end positioned near aperture 26 and a posterior end connected to a pump (not pictured). The pump can produce positive pressure to introduce fluids through the cannula, negative pressure to remove fluids through the cannula, or both. The pump can be external or internal to device 10. In some embodiments, device 10 further includes a controller to control activation and transfer rate of the pump. In some embodiments, the cannula can be deployed in and out of aperture 26 using an actuator similar to sliding actuator 16 described above. In some embodiments, the cannula can be a needle 24 having a lumen (not pictured).

As described elsewhere herein, the surgical devices of the present invention are able to enter the subretinal space under direct visualization using a deployable needle. The surgical devices therefore comprise at least two configurations: a retracted configuration wherein the tip of the needle is safely within a device, and a deployed configuration wherein the tip of the needle is exposed to pierce the eye/sclera to enter the subretinal space.

Referring back to FIG. 2A through FIG. 2C, the device 10 in a retracted configuration is depicted. In the retracted configuration, actuator 16 is in a posterior position within cutout 14. The tip of needle 24, located at the anterior end, is withdrawn within aperture 26.

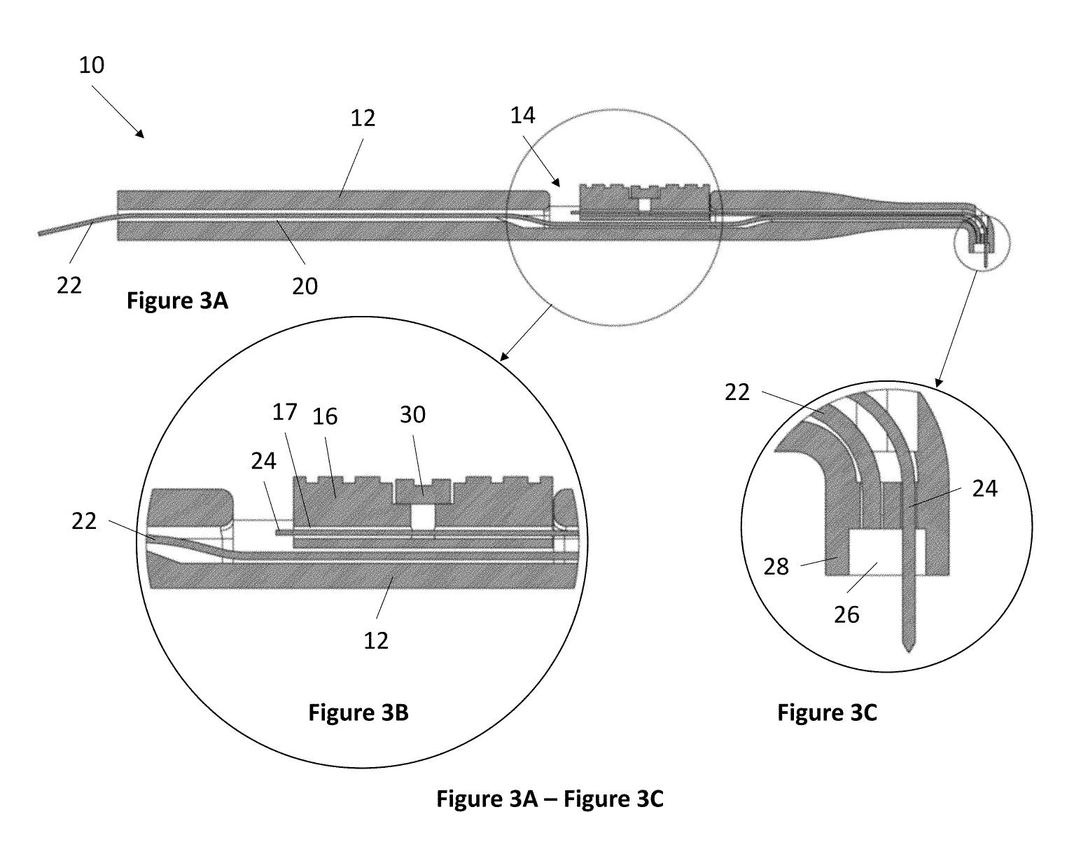

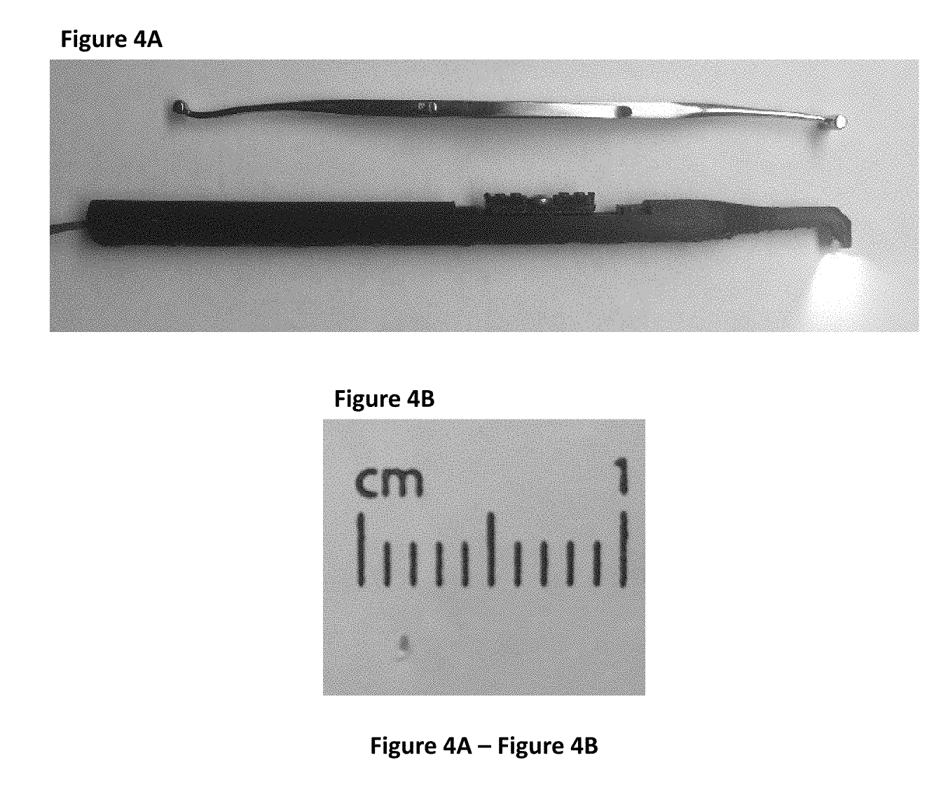

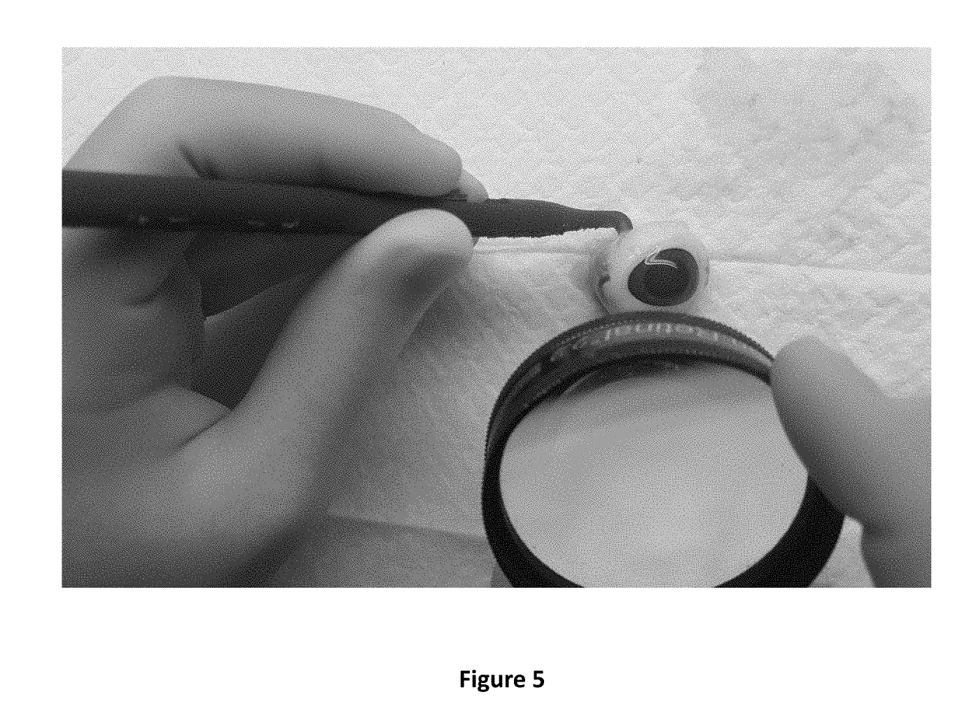

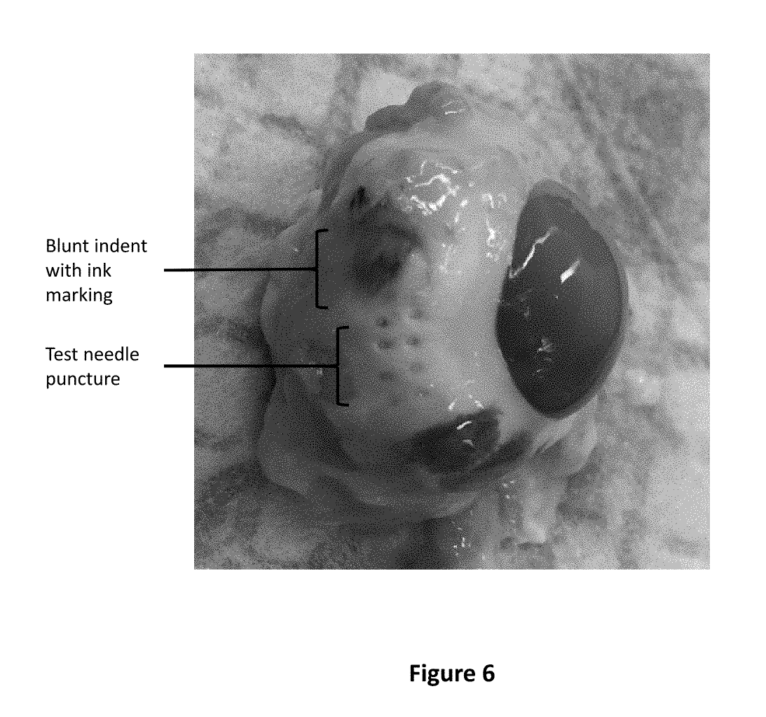

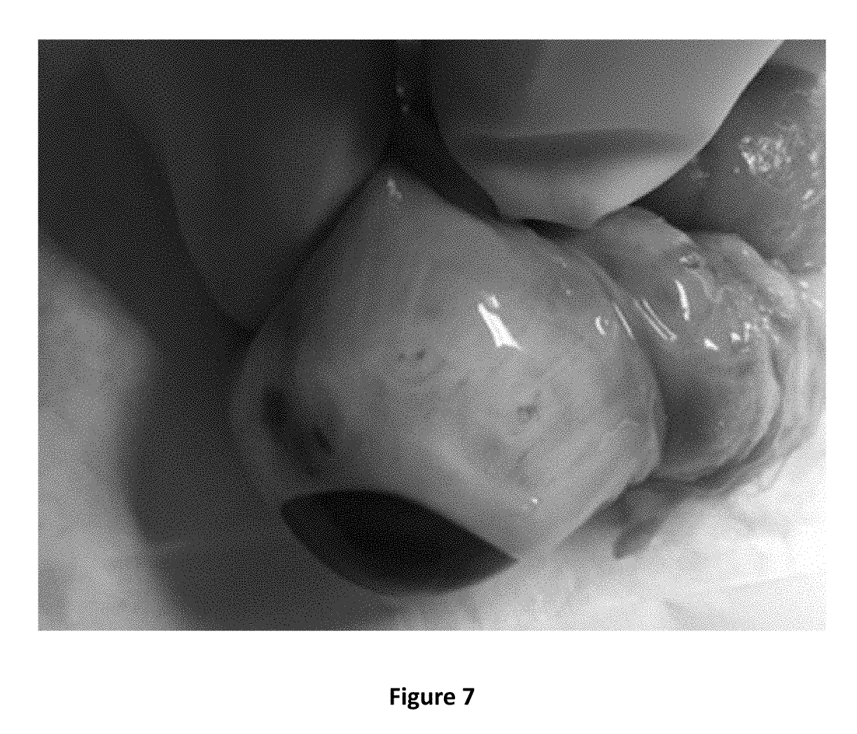

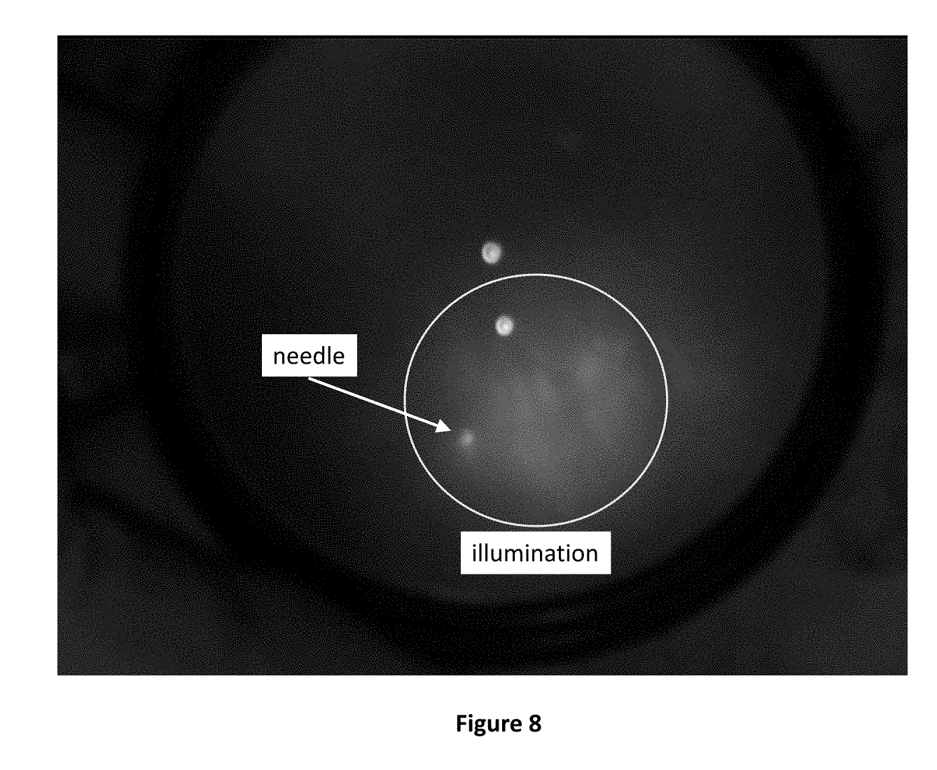

Referring now to FIG. 3A through FIG. 3C, the device 10 in a deployed configuration is depicted. To shift needle 24 from the retracted position to the deployed configuration, locking member 30 is engaged to fix the posterior end of needle 24 within actuator lumen 17, and actuator 16 is moved to an anterior position within cutout 14. The distance traveled by actuator 16 pushes needle 24 within lumen 20, and has the effect of extending the tip of needle 24 out of aperture 26 for the same distance. In certain embodiments, the length of needle 24 extending out of aperture 26 can be adjusted by disengaging locking member 30 from needle 24, relocating actuator 16, reengaging locking member 30 to needle 24, then moving actuator 16.

The aiming and placement of needle 24 are improved by way of the means of illumination provided by optical fiber 22. Placing aperture 26 against the sclera of a patient enables an operator to visualize from the interior of the eye the location of optical fiber 22 by any suitable means commonly used in the art (such as by indirect ophthalmoscopy or a wide angle contact lens with chandelier illumination). The means of illumination appears as a bright dot shining in from outside the sclera, and indicates the point of entry on the sclera for an extended needle 24.

The devices of the present invention can be made using any suitable method known in the art. The methods may vary depending on the materials used. For example, devices substantially comprising a plastic or polymer may be milled from a large block or injection molded. Likewise, devices substantially comprising a metal may be milled, cast, etched, or deposited by techniques such as chemical vapor deposition, spraying, sputtering, and ion plating. In some embodiments, the devices may be made using 3D printing techniques commonly used in the art.

The present invention also includes kits comprising the surgical devices described elsewhere herein. In some embodiments, the kits may comprise disposable surgical devices, wherein the entire surgical device may be thrown away after use. In other embodiments, the kits may comprise partially reusable surgical devices, wherein certain components may be discarded after use while other components may be retained and sterilized for further use. For instance, the kits may comprise a surgical device and one or more additional needles, wherein the one or more additional needles are disposable and may be replaced in the surgical device after each use. In other embodiments, the kits may comprise fully reusable surgical devices, wherein the entire surgical device is sterilizable by typical methods such as autoclaving, ultraviolet radiation, and the like.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out exemplary embodiments of the present invention, and are not to be construed as limiting in any way the remainder of the disclosure.

### Example 1: Preclinical Experiments in Porcine Eyes

Size and angle of the tip of the device: given the anatomic constraints of the eye within the orbit, the device must be of sufficiently small size to allow it to fit between the bony orbital wall and the eyeball in order for the device to access the proper location. Additionally, there is a curvature of at least 30 to 90 degrees of the tip of the device to allow the device to be held by the surgeon in the correct position and also allow the light and drainage needle to make contact with the proper portion of the sclera to allow drainage.

Marking the correct position on the sclera/eyeball. This is partially accomplished by the direct mechanical force of the device against the sclera, which dehydrates the scleral fibers to create a temporary mark. Iterations of the device were investigated to also apply a direct ink mark or a colored polymer to the surface of the sclera to allow more durable and longer lasting marking of the exact location of retinal breaks or subretinal fluid drainage sites on the eyeball during the surgical procedure.

Light source: a variety of vitreoretinal illumination fibers of varying size (20 gauge to 27 gauge) were utilized in device prototypes to determine optimal trans-scleral illumination. In preliminary experiments, an external 20 gauge light fiber on 100% illumination intensity on the Bausch and Lomb Millennium vitrectomy platform appeared to provide the ideal trans-scleral illumination. Additionally, varying the frequency of the light (i.e., turning on and off) provided a dynamic indication of the location of the tip of the device on the eye wall. Furthermore, changing the light wavelength to different visible colors of light, particularly red, can provide enhanced visualization of the tip of the device and highlight other features within the eye wall, including choroidal vessels and vortex veins, that are ideally avoided when entering the subretinal space to avoid hemorrhage.

Drainage needle: multiple design options were investigated in prototypes, including a stationary needle housed within a moveable shaft at the end of the device that functioned as a localizer in one position and then allowed for entering the subretinal space and for drainage in an unlocked position (i.e., stationary needle and a moveable cylinder shaft). Ultimately, the ideal drainage design was a surgeon-controlled, extendable nitinol needle that was malleable enough to traverse the 30-90 degree curve of the tip of the device and fashioned to be sharp enough to pierce the sclera and enter the subretinal space when actuated by a lever on the device by the surgeon. Active suction/aspiration controls allow further control of subretinal fluid drainage by the surgeon.

Cautery/diathermy: in order to decrease the risk of subretinal hemorrhage when piercing the sclera/choroid, the device can incorporate a ring that delivers cautery or diathermy to the scleral bed.

The device may be used in any vitreoretinal procedure in order to drain subretinal fluid externally, including but not limited to: primary scleral buckling procedures, rhegmatogenous retinal detachments repaired with pars plana vitrectomy alone, and serious retinal detachments. The device with the light alone, and without the drainage function, may also be used as an enhanced scleral depressor in a clinical setting to better localize retinal tears in the outpatient setting or for teaching of ophthalmology trainees or ophthalmology care extenders (optometrists, physician assistants, nurse practitioners, registered nurses) the technique of scleral depression.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A surgical device comprising:
an elongate shaft having a longitudinal axis, a posterior end, and an anterior end having an aperture that is recessed and encircled by a perimeter, wherein the anterior end and the aperture are curved away from the longitudinal axis at an angle of at least 30°;
a light source positioned within the aperture;
an actuator positioned along the length of the shaft; and
a needle connected to the actuator;
wherein the needle tip is extendable out of the aperture via activation of the actuator.

2. The device of claim 1, wherein the angle is between 30° and 120°.

3. The device of claim 1, wherein the angle is 90°.

4. The device of claim 1, wherein the perimeter forms a blunt scleral depressor.

5. The device of claim 1, further comprising a reservoir in the shaft connected to a delivery port near the aperture, wherein the reservoir stores a material selected from the group consisting of: an ink, a resin, and a polymer, and wherein the delivery port ejects the material stored in the reservoir.

6. The device of claim 1, further comprising connection means to a source of power.

7. The device of claim 6, wherein the source of power is a battery in the shaft.

8. The device of claim 6, wherein the source of power is external to the device.

9. The device of claim 1, further comprising diathermy means near the aperture.

10. The device of claim 1, further comprising cautery means near the aperture.

11. The device of claim 1, wherein the light source is an optical fiber having attachment means to an external source of light.

12. The device of claim 1, wherein the light source is at least one light emitting diode (LED) or laser diode.

13. The device of claim 1, wherein the light source is connected to a controller.

14. The device of claim 13, wherein the controller controls light activation, intensity, focus, diffusion, frequency, strobing, and color.

15. The device of claim 1, wherein the needle is constructed from a malleable material.

16. The device of claim 15, wherein the malleable material is nitinol.

17. The device of claim 1, wherein the connection between the needle and the actuator is adjustable by a locking member.

18. The device of claim 1, wherein the needle is removable.

19. The device of claim 1, further comprising a lumen having an anterior end near the aperture and a posterior end connected to a fluid pump.

20. The device of claim 19, wherein the fluid pump is controllable to generate a positive pressure, a negative pressure, or both.

21. The device of claim 19, wherein the lumen is positioned within the needle.
